(19) United States
(12) Patent Application Publication
(10) Pub. No.: US 2008/0312643 A1
(43) Pub. Date: Dec. 18, 2008

(54) TISSUE ABLATION SYSTEM INCLUDING GUIDEWIRE WITH SENSING ELEMENT

(75) Inventors: Wlodzimierz KANIA, Pointe-Claire (CA); Miriam LANE, Dollard-des-Ormeaux (CA); Sean CARROLL, Rancho Cucamonga, CA (US); Allan SKANES, London (CA)

Correspondence Address: CHRISTOPHER & WEISBERG, P.A., 200 EAST LAS OLAS BOULEVARD, SUITE 2040, FORT LAUDERDALE, FL, 33301, US

(73) Assignee: CRYOCATH TECHNOLOGIES INC., Kirkland (CA)

(21) Appl. No.: 12/199,255

(22) Filed: Aug. 27, 2008

Related U.S. Application Data
(62) Division of application No. 11/021,113, filed on Dec. 22, 2004.

(51) Int. Cl. A61B 18/02; A61B 18/12; A61N 7/02

(52) U.S. Cl. 606/21; 606/33; 601/3; 128/898

(57) ABSTRACT

A tissue ablation system for ablating tissue is presented having independent sensing and ablation capabilities. A sensing wire is positioned distally to the ablation region and passed through the ablation device allowing independent movement. The ablation device can ablate a substantial portion of a circumferential region of tissue. The tissue ablation system comprises an ablation device comprised of an elongated catheter with a proximal region and a distal region and an ablation element located proximate the distal region of the catheter. A sensing device having an elongated body with a proximal portion and a distal portion is adapted to be positioned within a vessel at or near a vessel ostium, wherein the sensing device is adapted to be slidably received within a lumen of the ablation device. The sensing device may be shaped in various configurations.

### CROSS-REFERENCE TO RELATED APPLICATION

This application is a divisional of Patent Application Serial No. 11021113, filed Dec. 22, 2004, entitled TISSUE ABLATION SYSTEM INCLUDING GUIDEWIRE WITH SENSING ELEMENT, the entirety of which is incorporated herein by reference.

### STATEMENT REGARDING FEDERALLY SPONSORED RESEARCH OR DEVELOPMENT

n/a

### FIELD OF THE INVENTION

The present invention relates to medical systems and more particularly to a movable sensor system for tissue ablation.

### BACKGROUND OF THE INVENTION

Many tissue ablation devices and methods have been developed for both diagnosis and for treating the various symptoms of abnormal heart rhythms, generally referred to as cardiac arrhythmias. The present invention is concerned with electrical isolation of anatomical structure, such as isolating the pulmonary veins from the left atrium for treatment of atrial fibrillation. Cardiac arrhythmias, and atrial fibrillation in particular, persist as common and dangerous medical ailments associated with abnormal cardiac chamber wall tissue and are often observed in the elderly.

Detailed examples of these ablation devices used for electrically isolating the pulmonary vein and methods for creating lesions are disclosed in U.S. Pat. Nos. 6,012,457 to Lesh; 6,164,283 to Lesh; 6,245,064 to Lesh; 6,245,599 to Lesh; 6,241,754 to Swanson; and 6,325,797 to Stewart.

Cardiac arrhythmias, including atrial fibrillation, may generally be detected using the global technique of an electrocardiogram (EKG). More sensitive procedures of mapping the specific conduction along the cardiac chambers have also been disclosed, such as, for example, in U.S. Pat. Nos. 5,500,011 to Desai; 5,657,755 to Desai; 5,555,883 to Avitall; 5,156,151 to Imran; 6,292,695 to Webster; and 6,064,905 to Webster. These devices are often coupled to an ablation device. For example, Patent Application No. WO 00/51683 (“the '683 application”) teaches the concept of using sensors mounted on an expandable member to achieve surface contact for mapping and ablation control. As has been described above, mapping using electrical signals identifies electrical isolation by comparing electrical signal propagation. The ideal ablation target may be the atrial tissue surrounding the Pulmonary Vein ostium. In such a situation, to adequately map, the electrodes should be positioned distal to the ablation location and inside the Pulmonary Vein, and not at the actual ablation site as taught in the '683 application.

With an increased emphasis on anatomical approaches to ablation and ablation at or near an ostium, there exists a need to de-couple the sensing technology used for mapping, from the ablation device such that the sensor does not obstruct the ablation member from engaging the tissue during the ablation procedure. Further, none of the above teaches the flexibility of using two devices with a single transceptal puncture to access the left atrium.

It is desirable, therefore, to provide a system that combines mapping and sensing capabilities with an ablation device wherein the sensing portion of the system is operated independently of the ablation portion and does not interfere with the ablation device in contact with the surface of the treated tissue.

### SUMMARY OF THE INVENTION

The present invention advantageously provides a method and system for ablating a circumferential region of tissue wherein a sensing wire is positioned distally to the ablation region and passes through the ablation device such that it may move with or independently of the ablation device without obstructing the surface-tissue interface.

In one embodiment, the present invention is a medical device having a sensor and a device body, wherein the sensor is movable with respect to the device body. In another embodiment, the invention comprises a method of positioning a sensor with respect to an ablation element wherein the sensor and ablation element are part of a single ablation device.

According to one aspect, the invention comprises a sensing device and an ablation device. The ablation device includes an ablation member that ablates a substantial portion of a circumferential region of human tissue such as the location where the pulmonary vein extends from the atrium. The ablation device includes an elongated body with a proximal end portion and a distal end portion. The ablation member is coupled to the elongated body such that the ablation member may be adjustable from a collapsed state to an expanded position. The adjustable ablation member is adapted to engage the substantial portion of circumferential region of tissue when in the expanded position.

According to another aspect of the present invention, a tissue ablation system is provided for ablating a region of tissue. The system comprises a treatment device, such as, for example, a probe or catheter, having a proximal region and a distal region and a treatment element located proximate the distal region of the treatment device. The system also includes a sensing device having a body with a proximal portion and a distal portion. The sensing device is preferably adapted to be positioned within a vessel and is adapted to be slidably received within a lumen of the treatment device.

According to another aspect of the invention, a tissue ablation system for ablating a region of tissue is provided. The system includes an ablation device comprised of an elongated catheter with a proximal region and a distal region and an ablation element located proximate the distal region of the catheter, and a sensing device having an elongated body with a proximal portion and a distal portion. The sensing device is positioned within a vessel and is adapted to be slidably received within a lumen of the ablation device. The sensing device is adapted to slidably track side by side with the ablation device through a sheath such that the ablation element maintains engagement with the tissue when the sensing device is slidably received within the lumen of the ablation device.

According to yet another embodiment or aspect of the invention, the invention comprises a sensing device having an elongated body with a proximal end portion and a distal end portion. The elongated body is adapted to be positioned within a vessel and positionable through another device. The distal end portion is configured to sense ECG signals in a circumferential region inside a vessel lumen.

According to still another aspect of the invention, the invention comprises a tissue treatment system for treating a region of tissue. The tissue treatment system comprises a treatment device comprised of an elongated catheter with a proximal region and a distal region and a treatment element located proximate the distal region of the catheter, and a sensing device adapted to be positioned within a vessel or at or near a vessel opening. The sensing device is adapted to be slidably received within a lumen of the treatment device, and the sensing device is also adapted to slidably track side by side with the treatment device through a sheath such that the treatment element maintains engagement with the tissue when the sensing device is slidably received within the lumen of the treatment device.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a side view of the tissue ablation device of the present invention;

FIGS. 2A-2C illustrate side views of the sensing device utilized in the present invention;

FIG. 3A is a side view of an alternate embodiment of the tissue ablation device of the present invention illustrating the use of the sensing device with a balloon catheter;

FIG. 3B is a side view of yet another embodiment of the tissue ablation device of the present invention illustrating the use of the sensing device with a balloon catheter; and

FIG. 4 is a side view of a further embodiment of the tissue ablation device of the present invention.

FIG. 5 is a side view of yet a further embodiment of the tissue ablation device of the present invention.

### DETAILED DESCRIPTION OF THE INVENTION

The present invention is a medical device that provides both electrical sensing and ablation capabilities in a single device. To insure that the ablation element provides sufficient circumferential contact with the target tissue ablation region, the sensing element of the device is positioned distally from the ablation element. In the preferred embodiment, the sensing element is a guide wire positioned within a lumen in the ablation device, comprises one or more electrodes. The electrodes can provide critical mapping information without hindering the ablation procedure, due to their location distally on the guidewire itself and not on the ablation element. Thus, the present invention provides a system that can allow for sensing and ablation procedures to be performed with only a single transceptal puncture.

As used herein, the term “cryogen” or “cryogenic fluid” refers to a fluid substance with properties suitable for: (i) steady flow through ducts of small diameter, (ii) high pressure compression into liquid phase, and (iii) evaporation and expansion to gas phase at low temperatures, typically at saturation temperature or in the range of −10 to −130 degrees centigrade. The cryogen may be any suitable, relatively inert “working fluid”, such as nitrogen, nitrous oxide, or carbon dioxide, or refrigerants such as chlorodifluoromethane, ethyl alcohol, or Freon (a trademark of DuPont), or any number of other refrigerants or fluids with a high thermal energy transfer capacity and low boiling point, as are commonly known to those skilled in the art.

Also as used herein, the term “catheter” refers to a medical device composed of any number of tubes and ancillary structures, for insertion into canals, vessels, passageways or other body cavities to permit the treatment of body tissue proximate to the catheter. A catheter may be constructed from a variety of suitable materials having a varying range of structural and thermal properties. It is understood that the particular structural, dimensional, and/or thermal properties of a catheter included in the present invention may considerably vary depending on the particular application of the device disclosed herein.

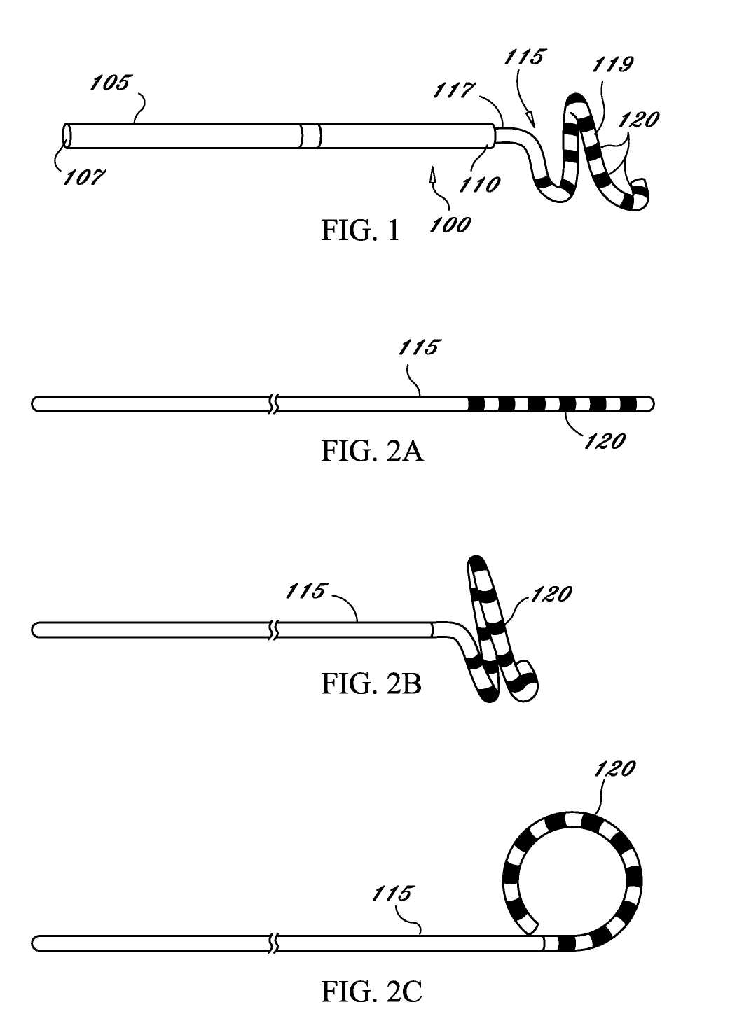

Referring now to the drawings, in which like reference designators refer to like elements, there is shown in FIG. 1 a tissue ablation device in accordance with the present invention, and designated generally as 100. An ablation device, such as a probe or a catheter 105, has an ablation member (catheter tip) 107 at its distal end, which may be used for various types of ablation procedures. The proximal end 110 of the catheter 105 is accessible to a surgeon and is connectable to a refrigerant source (not shown). The catheter 105 is preferably semi-rigid and flexible so as to be readily steerable to a desired location in a patient's body, in order, for example, to isolate the pulmonary vein from the left atrium in a patient's heart for treatment of such conditions as atrial fibrillation and cardiac arrhythmias.

The present invention may be used with all types of ablation catheters including cryocatheters and radiofrequency catheters. Catheters that carry out microwave, RF ablation, cool-tip RF ablation, thermal ablation and laser ablation procedures are also contemplated. In the preferred embodiment, the ablation device is a cryocatheter.

The ablation catheter 105 supplies cryogen to the desired location. The cryogen supplied may be either in a liquid or a gaseous state. The cryogen is cooled and/or compressed to a predetermined initial temperature and initial pressure before introduction into the catheter 105. The catheter 105 contains multiple inner tubes (not shown), preferably made of flexible or rigid material such a polymer, fiber, metal, or any combination thereof. The tubes are arranged to create a plurality of lumens (not shown) for the flow of cryogen therethrough. These lumens are arranged to create a circulation path for the flow of cryogen through the device. This includes an injection lumen (not shown) through which the cryogen is introduced into the catheter 105 to flow from a cryogen supply through to the ablation member 107, and a return lumen (not shown), through which cryogen eventually flows back to a controller unit from the catheter tip 107. The initial supply pressure of the cryogen is preferably on the order of 30 to 40 atmospheres, or 400 to 600 psia, much higher than the eventual final pressure in the vacuum return lumen. The resultant negative pressure gradient drives the high pressure cryogen drawn from the supply to flow through an injection lumen in catheter 105, to the catheter tip 107, and thereafter to flow back through the return lumen. Such catheter delivery systems are well known to those of ordinary skill in the art.

The ablation device is coupled to a sensing device having an elongated body with a proximal portion and a distal portion. The elongated body of the sensing device is typically between 0.014 inches to 0.042 inches in diameter and between 80 and 320 cm long, although this range is only an example and various-sized sensing devices may be used. The sensing device is positioned within a vessel and is adapted to be slidably received within a lumen in the ablation device. The sensor may, for example, be positioned at or near a vessel ostium. The sensing device can detect pressure, electrical activity, temperature or other characteristics such as impedence, necessary to provide mapping data to a user, in order to perform ablation procedures.

The sensing device preferably contains one or more electrodes 120 disposed about its exterior surface. One example of a sensing device compatible with the present invention is a guide wire 115. Catheter 105 is guided to the desired treatment site via guide wire 115. Referring to FIG. 1, guide wire 115 has a distal end 117 and a proximal end 119. Guide wire 115 is used to manipulate the catheter 105 through the patient's body to the ablation site. The guide wire 115 and the catheter 105 may be positioned within a vessel to ablate a substantial portion of the circumferential region of tissue at or near the location where the pulmonary vein extends from the atrium. The guide wire 115 is distal from catheter 105 and is slidably received within a lumen in catheter 105. Guide wire 115 can be separately controlled to move with or independently from catheter 105.

One or more electrodes 120 are positioned circumferentially around guide wire 115. Electrodes 120 provide mapping and sensing capabilities and are positioned distal from catheter 105 to assure that the sensing device does not interfere with catheter tip 107. Because guide wire 115 is slidably received within catheter 105, and is positioned distally from catheter 105, the guide wire does not obstruct the interface between the ablation member and the target surface tissue.

FIGS. 2A-2C illustrate various embodiments of guide wire 115. FIG. 2A illustrates guide wire 115 in a generally straight, circumferential shape located at the distal end of catheter 105 (not shown). The circumferential shape can be formed by various methods including inserting a pre-shaped inner member comprised of shape-memory material within the guide wire, activating a pull wire, or by removal of a stylet or other means known to those skilled in the art.

FIGS. 2B and 2C illustrate two of the various shapes that can be formed by controlling guide wire 115 to contact human tissue in various locations in the body. Once again, electrodes 120 can be positioned so as to contact tissue in difficult-to-reach locations in the patient in order to provide mapping information for ablation procedures. Various loops and circular configurations can be formed to allow electrodes 120 on guide wire 115 to touch the desired tissue region, for example the pulmonary vein or coronary sinus wall, in a number of locations around the circumference of the vein.

In an alternate embodiment, the guide wire 115 can be independently controlled and adjusted from a first, straight state, to a second, coiled orientation to allow electrodes 120 to radially contact the tissue of a blood vessel wall. For example, the sensing device 115 may be comprised of expandable, “balloon-like” material with the electrodes 120 disposed on the balloon. The balloon can be expanded to contact the vessel wall in a number of different locations to perform mapping procedures.

The catheter 105 may be pre-shaped to circumferentially engage the vessel wall or deflected to engage the vessel wall. Methods such as the use of a pull-wire may be used to cause the ablation device 105 to deflect to produce various shapes. By deflecting the ablation device, a catheter 105 may be re-directed in more than one direction in a single plane, as well as in more than one plane, to engage tissue in the target ablation region.

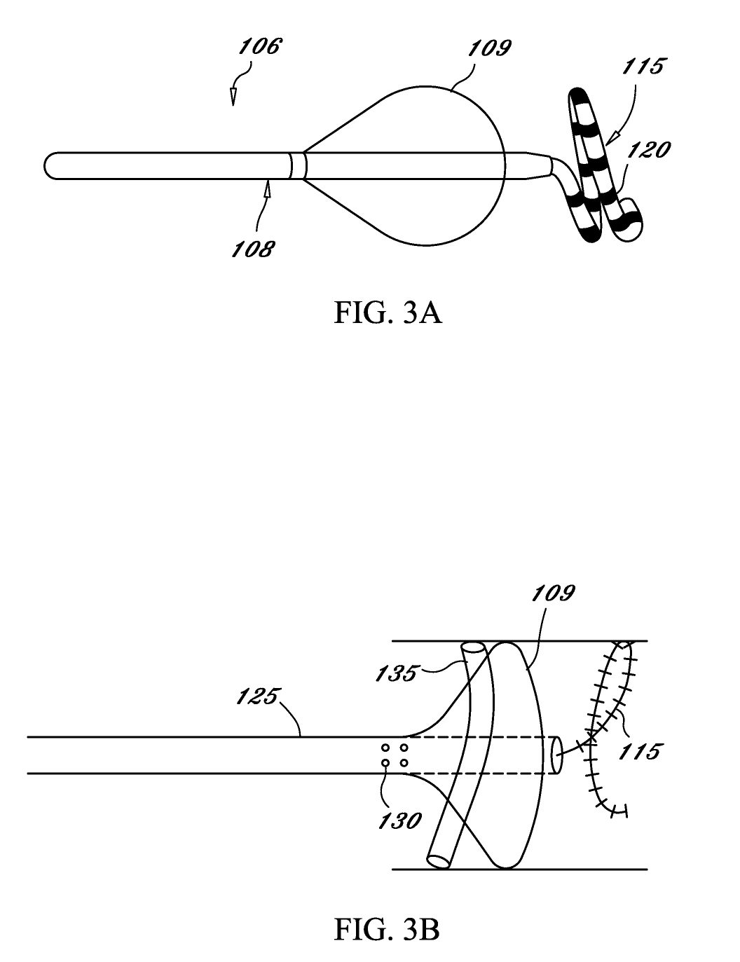

In one embodiment of the present invention, catheter 105 may be adjusted between a radially collapsed configuration and a radially expanded configuration. As described above for the sensing device, the ablation device may also be comprised of balloon-like material. FIG. 3A illustrates a balloon catheter 106 coupled to a guide wire 115 having sensing electrodes 120 around its outer circumference. Balloon catheter 106 has one or more expandable balloon portions 109 to engage the tissue of the patient at or near the vessel ostium or inside a vessel. The balloon portion 109 maintains its engagement with the tissue while the sensing device is slidably received within the lumen of the balloon catheter 106.

The specific size and shape of the balloon portion 109 may be determined prior to use to best fit the targeted vessel where an ablation or treatment procedure is to be performed. Balloon catheter 106 is inflated so that a balloon portion 109 contacts the inner walls of the blood vessel proximate the ablation area. The balloon portion 109 is comprised of a flexible, expandable membrane and is coupled to a catheter tube 108, wherein the balloon catheter 106 is guided to the desired treatment site via guide wire 115. The particular shape of the expanded balloon portion 109 may be predetermined by the use of a preformed balloon membrane, a memory retaining material, or other structural attribute wherein the expanded balloon portion 107 is configured to form a particular shape, yet also remain somewhat conformable.

FIG. 3B illustrates another embodiment of the present invention. In this embodiment, a sheath 125 is provided with a compliant, inflatable balloon portion 109 on its distal end. The flexible balloon portion 109 at the distal end of the sheath allows for the forming of different shapes within the vessel. Side holes 130 may be provided proximal to balloon portion 109 to allow for perfusion through the center of the balloon. This allows the balloon to remain inflated and to maintain perfusion throughout the ablation process performed by the AC cooling segment 135.

The benefit of the embodiment depicted in FIG. 3B is that the heat load flowing through the vessel to the target tissue is diminished due to the effects of the inflated balloon 109. Cooling segment 135 can now freeze the target tissue more effectively due to the reduced heat load and more efficient heat transfer to the target tissue.

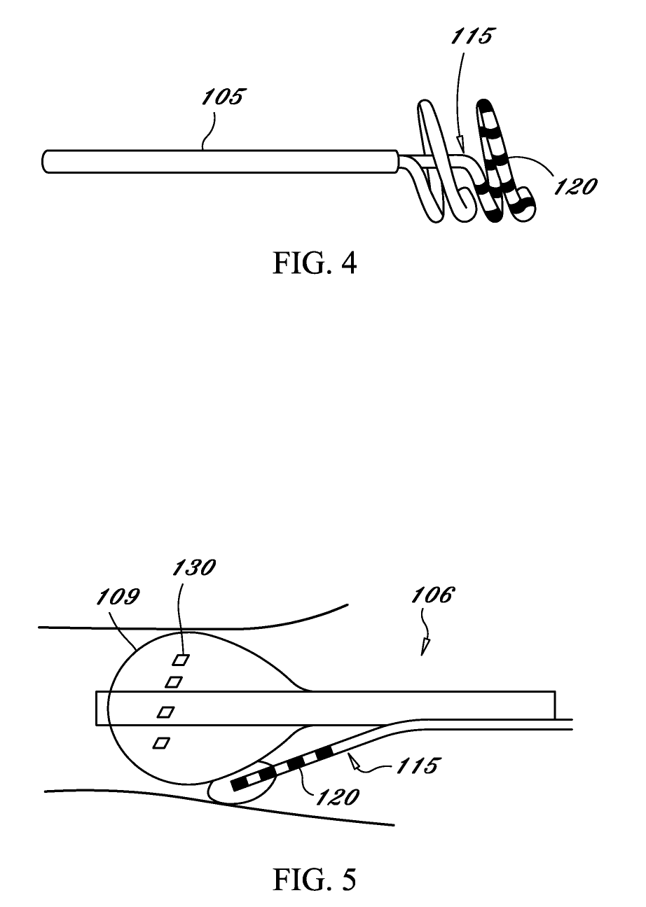

FIG. 4 shows a further embodiment of the present invention. Catheter 105 forms the shape of a loop at its distal end. Guide wire 115 passes through the distal loop portion of catheter 105. By employing differently shaped sensing devices and ablation devices a series of independently controlled mapping and ablation procedures can take place. The present invention allows for independent control of each procedure while maintaining the sensing device at a distance from the ablation device. In this fashion the sensing device, or guide wire, which passes through the interior portion of the ablation device, does not interfere with the catheter tip's engagement with the vessel wall during the ablation procedure.

FIG. 5 illustrates yet another embodiment of the present invention. A balloon catheter 106 is coupled to a guidewire 115 having one or more electrodes 120. In this embodiment, the expandable portion of the balloon catheter acts to decrease blood flow through a cavity while at least one electrode detects electrical activity, both of which act to facilitate cryoablation. Side holes 130 may be provided proximal to balloon portion 109 to allow for perfusion through the center of the balloon. This allows the balloon to remain inflated and to maintain perfusion throughout the ablation process.

The present invention is equally adaptable with various different types of ablation devices including but not limited to microwave, ultrasound and RF ablation elements, cryogenic ablation elements, thermal ablation elements, light-emitting ablation elements, ultrasound transducers and other substance delivery elements.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A method of treating tissue, comprising:
positioning a treatment device proximate tissue to be treated;
slidably positioning a sensing device with respect to the treatment device; and
detecting an electrical activity of the tissue with the sensing device.

2. The method of claim 1, further comprising cooling the tissue with the treatment device.

3. The method of claim 1, further comprising ablating the tissue with the treatment device.

4. The method of claim 3, wherein ablating the tissue is achieved at least in part by circulating a cryogenic fluid through the treatment device.

5. The method of claim 3, wherein ablating the tissue is achieved at least in part by transmitting radiofrequency energy from the treatment device.

6. The method of claim 3, wherein ablating the tissue is achieved at least in part by transmitting ultrasonic energy from the treatment device.

7. The method of claim 1, wherein positioning the treatment device includes inflating a balloon on the treatment device.

8. The method of claim 1, wherein positioning the treatment device includes engaging a circumferential portion of the tissue with a portion of the treatment device.

9. The method of claim 1, wherein positioning the treatment device includes inserting at least a portion of the treatment device into a blood vessel.

10. The method of claim 1, wherein the tissue to be treated is cardiac tissue.

11. The method of claim 1, wherein slidably positioning the sensing device includes slidably moving at least a portion of the sensing device through a lumen within the treatment device.

12. The method of claim 1, further comprising repositioning the treatment device based at least in part on the detected electrical activity.

13. The method of claim 1, further comprising transitioning the sensing device from a substantially straightened configuration to a substantially circumferential configuration.

14. The method of claim 1, further comprising transitioning the sensing device from a substantially straightened configuration to a substantially coiled configuration.

15. A method of treating tissue, comprising:
positioning a treatment device proximate cardiac tissue to be treated;
slidably positioning a sensing device through at least a portion of the treatment device;
detecting an electrical activity of the tissue with the sensing device; and
ablating the cardiac tissue with the treatment device.

16. The method of claim 15, further comprising cooling the cardiac tissue with the treatment device prior to ablating the cardiac tissue.

17. The method of claim 15, wherein ablating the tissue is achieved at least in part by circulating a cryogenic fluid through the treatment device.

18. The method of claim 15, wherein treatment device is a balloon catheter.

19. The method of claim 18, wherein positioning the treatment device includes engaging a circumferential portion of the tissue with a portion of the treatment device.

20. The method of claim 15, further comprising transitioning the sensing device from a substantially straightened configuration to a substantially circumferential configuration.

21. A method of treating tissue, comprising:
positioning a balloon catheter proximate cardiac tissue to be treated;
slidably positioning a sensing device through at least a portion of the catheter;
transitioning the sensing device from a substantially straightened configuration to a substantially circumferential configuration;
detecting an electrical activity of the tissue with the sensing device; and
ablating the cardiac tissue with the treatment device.

22. The method of claim 21, wherein ablating the tissue is achieved at least in part by circulating a cryogenic fluid through the balloon catheter.
